(19) United States
(12) Patent Application Publication
(10) Pub. No.: US 2021/0236763 A1
(43) Pub. Date: Aug. 5, 2021

(54) SLEEPING AID DEVICE

(71) Applicant: Imraan Feisal JHETAM, Exeter (GB)

(72) Inventor: Imraan Feisal JHETAM, Exeter (GB)

(21) Appl. No.: 17/166,562

(22) Filed: Feb. 3, 2021

(30) Foreign Application Priority Data
Feb. 4, 2020 (GB) 2001483.3

(51) Int. Cl. A61M 16/10; A61M 21/02; A61M 16/06; A61B 5/1455; A61B 5/00; A61M 16/12; A61B 5/11; A61M 16/20; A61B 5/08

(52) U.S. Cl. CPC A61M 16/1005; A61M 21/02; A61M 16/0683; A61B 5/14552; A61B 5/6803; A61B 5/4836; A61M 2202/0266; A61B 5/1114; A61M 16/105; A61M 16/208; A61B 5/082; A61M 2202/0208; A61M 2202/0225; A61M 16/12

(57) ABSTRACT

The present invention relates to a device adapted to be worn on a user's head to aid sleeping. The sleeping aid device comprises: a head strapping; a nose and mouth mask; and electronic apparatus to detect trigger events for reducing or increasing the oxygen in a supply of air to the wearer of the mask.

### FIELD OF THE INVENTION

The present invention relates to a device adapted to be worn on a user's head to aid sleeping.

### BACKGROUND

Increasingly sleep is recognized as important for mental well-being by many societies. In particular lack of sleep may make many people anxious, mentally disturbed or subject to health concerns. However many such people may suffer from an inability to wind down from stress-inducing or busy lifestyles, meaning that many find it difficult to attain sleep in the first place.

Oxygen saturation levels in the blood can have a powerful effect on whether the body feels sleepy.

### PRIOR ART

The present invention arose in order to overcome problems suffered by existing devices.

### SUMMARY OF THE INVENTION

According to a first aspect of the invention there is provided a device adapted to be worn on a user's head to aid sleeping comprising: head strapping; a nose and mouth mask; and electronic apparatus to detect trigger events for reducing or increasing the oxygen in a supply of air to the wearer of the mask.

According to a second aspect of the invention there is a device adapted to be worn on a user's head to aid sleeping, the device comprises: a mask that defines part of an airway, the mask encloses a breathing space and includes at least one sensor which senses at least one trigger event and an oxygen regulator which is operative to vary an oxygen flow from an oxygen supply in response to a sensed at least one trigger event, wherein the sensed at least one trigger event causes the oxygen regulator to adjust an amount of oxygen in the breathing space.

The oxygen supply may be oxygen in ambient air outside the device when it is worn on a user's head. The oxygen regulator may be adapted to draw oxygen from air.

The at least one sensor may be related to providing information or data on atmospheric conditions or may be related to a wearer or user of the device adapted to be worn on a user's head to aid sleeping. For example the sensor or sensors may be arranged to control oxygen reduction or increase relative to the ambient air supply and comprise a central controlling element of the apparatus.

The at least one sensor may be or may comprise an oxygen sensor. The oxygen sensor may be or may include an oximeter and the at least one trigger event is dependent upon a blood oxygen concentration level of the user. The oximeter may be a pulse oximeter. Pulse oximetry is a non-invasive and painless test that measures oxygen saturation level, or the oxygen levels in blood.

The oximeter may consequently be arranged to provide detection of trigger events, for example excessive or insufficient levels of oxygen.

It may be that the device adapted to be worn on a user's head to aid sleeping knows when to restrict by constantly or intermittently monitoring blood oxygen level with a nasal oximeter, which may be adapted as a sensor of blood oxygen level. This nasal pulse oximeter may attach to the bridge of the nose, for example pushed against the user's nose using the securement mechanism of a resiliently deformable material.

The oximeter may be provided on the mask, or head strapping so as to contact the user. The mask may comprise a resiliently deformable portion and means to retain the mask on the user's head. The mask may include a bridge portion which bridges the user's nose, and the oximeter is retained in the bridge portion and is adapted to be pressed against a nose of user. So the oximeter may be held in place on the user using a securement mechanism. Such securement mechanism may comprise pressure, adhesive and/or positioning parts.

In some embodiments the device adapted to be worn on a user's head to aid sleeping comprises a gas probe. The gas probe may be disposed in an inner of the mask to measure concentration of gas in the breathing space. The probe may be adapted to measure oxygen, nitrogen and/or carbon dioxide concentration within the mask. So, the at least one sensor may be or include a gas probe to measure levels of a gas within the breathing space, the gas being from the group comprising: argon, carbon dioxide, nitrogen and oxygen, and the at least one trigger event is dependent upon a concentration level of the measured level of gas.

For example in some embodiments the device adapted to be worn on a user's head to aid sleeping will gradually reduce the blood oxygen level to approximately 90% saturation. When sleep is detected via an orientation sensor, the device will allow the blood oxygen saturation level to increase back to 95% or more, which is the norm. The (blood oxygen saturation) SpO2 level is monitored via the paranasal pulse oximeter and the gas probe within the mask may also monitor the (O2) oxygen, (CO2) carbon dioxide and nitrogen levels as an extra precaution.

In some embodiments the device adapted to be worn on a user's head to aid sleeping comprises the orientation sensor. This orientation sensor may detect orientation of the user's head while the user is wearing the device. The least one sensor may be an orientation sensor to detect an orientation of the user's head and the at least one trigger event may be an orientation of the user's head. The orientation of the user's head may be associated with a risk factor. The trigger events identified may affected by the risk factor. For example when sleeping face down against a bed or pillow.

In some embodiments the orientation sensor comprises an accelerometer. In other embodiments in addition or the alternative the orientation sensor comprises a gyroscope.

In some embodiments the device adapted to be worn on a user's head to aid sleeping comprises a battery energy source. In this way it may be envisaged that there is no need for external wiring or an external power source, meaning the device may be comfortably worn in a bed situation without interference.

In some embodiments the battery energy source is displaceable for charging or replacement purposes. In some embodiments the battery energy source is rechargeable. The device adapted to be worn on a user's head to aid sleeping may comprise an induction charging mechanism, or a socket for insertion of a charging cable such as a micro-USB plug.

In some embodiments the device adapted to be worn on a user's head to aid sleeping may be connected in use to an oxygen concentrator which may ionize the oxygen.

Said oxygen concentrator may be connected to the mask by a cleanable or disposable air tube in use. The concentrator may be locally situated, for example located on the mask. The oxygen regulator may comprise the oxygen concentrator.

The device adapted to be worn on a user's head to aid sleeping may comprise at least one filter. The oxygen regulator may comprise the at least one filter. Ideally the oxygen regulator and/or at least one of the filters is arranged to operate in response to a trigger event. The oxygen regulator and/or the first filter may be disposed in a first passage to filter ambient air into the breathing space in response to a trigger event. The filter may preferentially allow oxygen pass and thereby act as the oxygen concentrator.

The filter may have a semi-permeable membrane that is permeable to oxygen and non-permeable to at least one other gas which constitutes air. The at least one filter may have a semi-permeable membrane that is non-permeable to oxygen and permeable to at least one other gas which constitutes air. The filter may be an oxygen membrane filter. Ideally the membrane is only partially non-permeable to oxygen so that the membrane favor passage of other gases in air and restricts but does not completely block passage of oxygen. So there is always a safe mixture of breathing gas in the breathing space.

Alternatively or additionally the device may use microporous materials or zeolites that can selectively adsorb or pass molecules in a designated range of sizes, for example a zeolite that selects for oxygen and/or argon and use pressure swing adsorption to make the concentrated oxygen stream. The at least one filter may comprise a zeolite material that absorbs oxygen and is does non absorb at least one other gas which constitutes air. The at least one filter may comprise a zeolite material that is non-absorbent to oxygen and absorbs at least one other gas which constitutes air.

The device adapted to be worn on a user's head to aid sleeping may comprise at least one one-way valve. Such valve or valves may be controlled electrically in response to a trigger event. Such valve or valves may refer to both inlet or intake and exhaust or outlet valves. Ideally at least one of the inlet valves and at least one of the outlet valves is only partially closable. In this way a safe supply of breathing gas including oxygen is always ensured in the breathing space. The oxygen regulator may comprise the inlet and/or outlet valves and be capable of increasing and/or decreasing the amount or concentration of oxygen in the breathing space.

The oxygen regulator may comprise a first valve which is operative in response to a trigger event to regulate intake of filtered ambient air into the breathing space. The first valve is a one-way valve that prevents flow of air out of the breathing space. The first valve and a first filter are disposed in a first passage to filter ambient air to regulate flow of oxygen into the breathing space in response to a trigger event. So the concentration of oxygen in the breathing space may be regulated using feedback from the sensors to identify the trigger event. The oxygen concentrator may comprise the valves.

There may be a second inlet valve. The first and second valve may be disposed at first and second entrances of the passage respectively. Each valve may have a respective filter in the flow stream of that valve to control oxygen let in by that valve. The first and second entrances may be at opposed ends of a passage or may each be in their own individual passage.

The device adapted to be worn on a user's head to aid sleeping may comprise a one-way exhaust or outlet valve. The outlet valve may provide an outlet for and restrict or control exhaled air leaving the breathing space device. The at least one filter may include a second filter in a second passage to divert exhaled air from the breathing space in response to a trigger event. So the concentration of oxygen in the breathing space may be adjusted by controlling both flow into and/or out of the breathing space.

One or more of the valves, filters and/or concentrators may be disposed in an air way mask or in an oxygen regulator or a cartridge connected to the air way mask.

In this way the device adapted to be worn on a user's head to aid sleeping restricts new oxygen from entering the breathing space within the mask with oxygen filtering and a one-way entrance valve which valve only allows air in, and/or allows control of or restricts exhaled air from leaving. This is to reduce exhaled air to be able to tweak the O2 in the CO2, O2, nitrogen mix in the mask.

The device adapted to be worn on user's head to aid sleeping may comprise an electronic trigger event identification apparatus arranged to identify trigger events from signals it receives from the sensors. The trigger event identification apparatus may be arranged to operate the oxygen regulator, filters and valves according to the trigger events. This valve or these valves may be controlled digitally. For example the one or more valves may comprise a semi-permeable membrane valve that is controlled or opened by applying current. In some embodiments the device adapted to be worn on a user's head to aid sleeping comprises a transceiver. In this way the device may be controlled remotely by a wirelessly or network enabled item such as a smartphone or other personal computing item. The device adapted to be worn on a user's head to aid sleeping may be controlled, and information gathered, updated or data transferred via the transceiver. The oxygen regulator may comprise the trigger event identification apparatus.

The device adapted to be worn on user's head to aid sleeping may comprise a self-contained part including the trigger event identification apparatus, at least one of the sensors, the oxygen regulator, at least one of the valves, and/or at least one of the filters. The self-contained part may be connected to the nose and mouth cover of the airway mask. It may be displaceable from the nose and mouth cover of the airway mask. It any be connected to the mask nose and mouth cover by a connection which allows it to rotate freely on the cover for the comfort of the user while sleeping. The self-contained part may be arranged to displace from the nose and mouth cover for maintenance or cleaning purposes. Alternatively or additionally the self-contained part may be displaced and/or rotated on the nose and mouth cover for comfort purposes for example according to user preference and/or sleeping habits.

The self-contained part may have a form of a detachable cartridge which is affixed to a nose and a mouth cover of the airway mask, the cartridge comprising at least one filter and at least one sensor. The cartridge may comprise a first valve to regulate intake of filtered ambient air into the breathing space in response to a trigger event, and/or comprising a second valve to regulate outflow of breathed air from the breathing space according to the trigger events. The cartridge connects to a nose and mouth cover of the airway mask on a mounting which opens to allow the cartridge to receive ambient air external of the device.

The invention will now be described by way of example only and with reference to the Figures in which:

### BRIEF DESCRIPTION OF FIGURES

FIG. 1 shows an isometric view of an embodiment of a device adapted to be worn on a user's head to aid sleeping;

FIG. 2 shows a reverse isometric view of the device adapted to be worn on a user's head to aid sleeping shown in FIG. 1;

FIG. 3 shows an exploded isometric view of the device adapted to be worn on a user's head to aid sleeping shown in FIG. 1;

and

FIG. 4 shows a reverse isometric exploded view of the device adapted to be worn on a user's head to aid sleeping shown in FIG. 1.

### DETAILED DESCRIPTION OF FIGURES

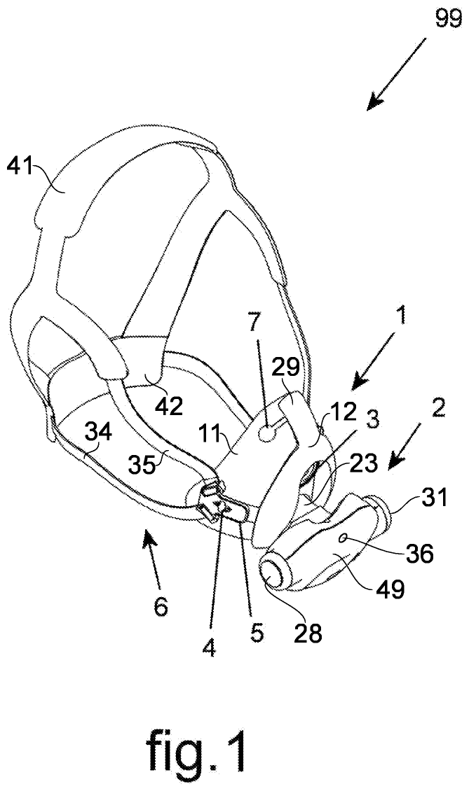

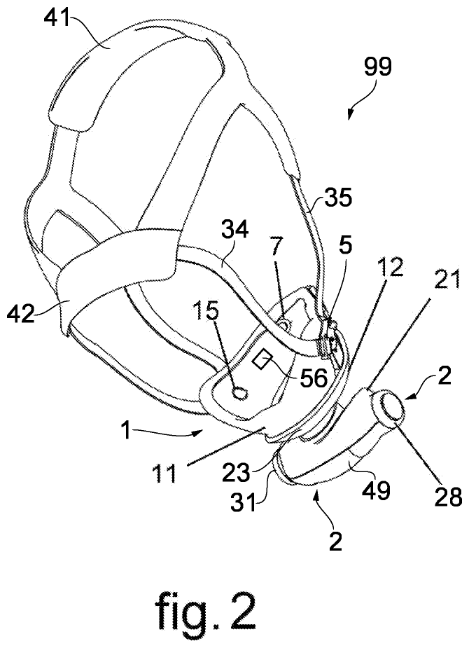

With reference to the figures there is shown an embodiment of a device adapted to be worn on a user's head to aid sleeping 99. FIG. 1 and FIG. 2 shows that the includes an airway mask 1 in a form of a nose and mouth cover. A head strapping 6 has straps 34, 35, 41, 42 which in use go over and behind a user head to hold the air way mask over the user's nose and mouth while their head is turning as they are sleeping with their head resting on a bed.

The airway mask 1 defines part of an airway 28, 36, the mask encloses a breathing space and includes at least one sensor 7, 26 which senses at least one trigger event and an oxygen regulator 3, 22, 27, which is operative to vary an oxygen flow from an oxygen supply in response to a sensed at least one trigger event, wherein the sensed at least one trigger event causes the oxygen regulator to reduce or to increase an amount of oxygen in the breathing space.

An elongate cartridge 2 is mounted sideways onto the front of the nose and mouth cover so that the elongate ends 28, 31 of the cartridge 2 extend sideways to opposite sides of the mouth of the user wearing the air way mask 1. There are entrances to ambient air at the elongate ends 28, 31 for the user wearing the airway mask to breathe through.

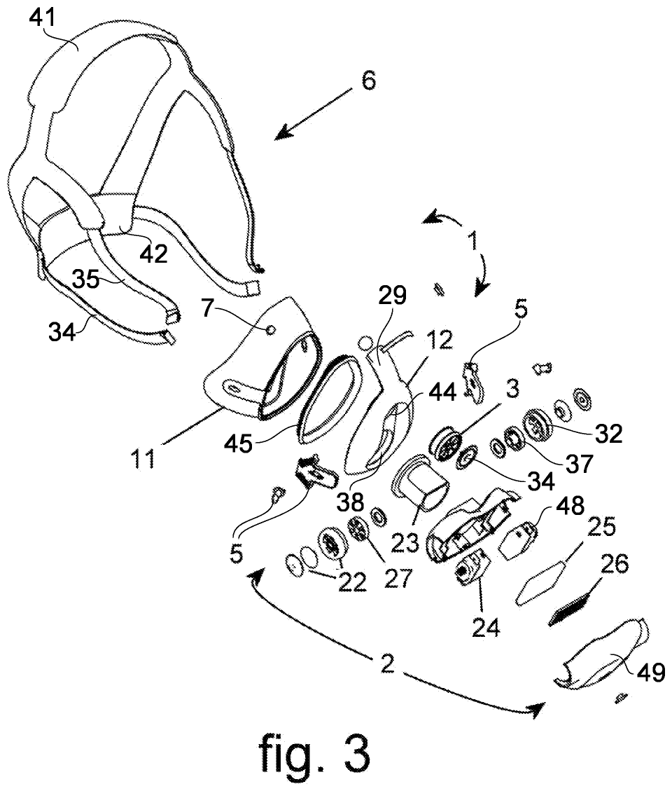

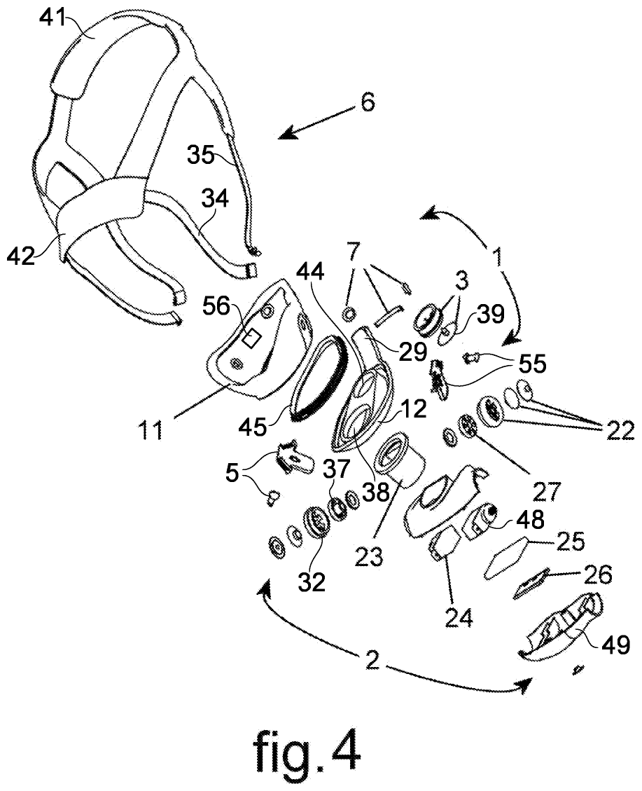

FIG. 3 and FIG. 4 are exploded views showing internal components of the airway mask 1 and cartridge 2 to detect trigger events for reducing or increasing oxygen supply to the wearer of the mask 1. The cartridge 2 has a housing 49 to cover internal components. The cartridge also comprises components of the oxygen regulator.

The airway mask 1 comprises a hypo-allergenic inner 11 located in use against the user's face and specifically under the chin, against the cheeks, and over the nose bridge. The inner has a shape of a nose and mouth cover. The inner 11 comprises a polymeric material that is resilient and flexible to seal airtight to the user's face. The inner 11 may comprise silicone inner although other materials may be used so that the inner 11 may be located in use against the user's face for comfort.

The inner 11 has a front opening over where the user's mouth and nose are located in use. There is a rim around the front opening which is covered by an outer 12 of the airway mask 1. The outer 12 fits over the front of the inner 11. The outer 12 has a form of an upper lip and under nose extension and comprises a material which is relatively more rigid than the inner 11. The outer 12 maintains the shape of the airway mask 1. An oval gasket 45 intermediate the inner 11 and the outer 12 makes an airtight seal ensuring gas may only enter and exit the breathing space through orifices through the outer 12. These are an inlet orifice 38 and outlet orifice 44.

The cheek sides of the inner 11 provide pivot points 15 for hitches 5, 55 for the head strapping 6. Such hitches 5, 55 comprise bifurcated clip means for the head strapping which comprises two bands 34, 35.

The hitch further includes a quick release slot 4. The quick release slot 4 provides a means allows for the hitch 5 to be rotated in reference to the pivot points 15, so as to allow the pivot points 15 to exit the slots 4 in the hitch 5.

The head strapping 6 comprises two flexible band members 34, 35 extending from the hitch diagonally rearwards in use and connected to harness with padding. The two flexible band members 34, 35 diverge from the hitch 5 so they go above and below wearer's ears and then connect to the back of the head straps 41, 42.

The head strapping 6 may be envisaged to comprise inelastic or elastic webbing 34. 35, 41, 42. The webbing may be coated or padded with a soft comfortable material that slides easily the head. For example a silicone padding, silicone and foam straps may be used, although these materials are not essential. The head strapping is adapted to securely hold the airway mask 1 in place whilst at the same time ensure maximum comfort for undisturbed sleep.

A gas probe 56 is disposed on the inside of the inner 11 to measure concentration of gas in the breathing space. The probe 56 is adapted to measure oxygen, nitrogen and carbon dioxide concentration.

The air mask 1 comprises a nasal oximeter 7 which is located in use over the bridge of the mask or user's nose. The nasal oximeter 7 is disposed in the nose and mouth cover inner 11. A contact surface of the nasal oximeter 7 is exposed to the interior of the inner 11. The contact surface is urged against the user's nose or cheek when the head strapping 6 pulls the nose and mouth cover inner 11 sealed against the user's face. Light from the oximeter shines directly into the user's skin to measure a blood oxygen level.

A level of oxygen in the blood detected by the oximeter 7 that is over/under a limit triggers the components in the cartridge 2 to provide an amount or concentration of breathing gas into the nose and mouth cover for the user to breath. There is a trigger event when the level of oxygen in the blood goes over/under the limit. So trigger events include events detected by the nasal oximeter 7.

The oximeter 7 and gas probe 56 are in communication with a trigger event identification apparatus 26 which identifies trigger events detected by these sensors.

The cartridge 2 containing an apparatus of components for oxygen regulation and detecting and processing trigger events is connected by a hollow mounting tube 23 for the breathing gas to be inhaled through. The hollow mounting tube 23 extends from about halfway between the ends 29, 31 of the elongate cartridge perpendicular to the elongate axis of the cartridge.

The cartridge 2 is mounted on the air mask 1. There is an inlet orifice 38 through the outer 12 which receives the hollow mounting tube 23. The cartridge is connected to mounting tube so the cartridge is allowed to rotate relative to the airway mask 1 to accommodate the user's comfort according to preference or position while they are sleeping with their head on a bed. The breathing entrances 28,31 at the elongate ends of the cartridge rotate to positions where they are unblocked by the bed or a pillow.

There is at least one orientation sensor 26 in the cartridge 2. The orientation sensor 26 comprises accelerometer and/or a gyroscope. The orientation sensors are adapted to detect predefined movement patterns indicating sleep and/or predefined movements indicating sleeplessness. There is a trigger event when the predefined movements indicating sleep are detected. So trigger events include events detected by the orientation sensor.

The orientation sensor 26 is combined with a trigger event identification apparatus 26 in the cartridge 2. The trigger event identification apparatus 26 is arranged to communicate with the oximeter 7 and the orientation sensor 26 so that the trigger events are identified as they occur.

The cartridge 2 comprises a first one-way inlet valve 22 at the entrance 28 and a second one-way inlet valve 32 at the opposite entrance 31. These inlet valves allow air to flow into the cartridge 2 as the wearer breathes in through the hollow mounting tube 23. The inlet valves prevent air from flowing out.

The cartridge 2 comprises a first inlet filter 27 between the first one-way inlet valve 22 and the hollow mounting tube 23. The cartridge 2 comprises a second inlet filter 37 between the second one-way inlet valve 32 and the hollow mounting tube 23.

The two valves 22 can be opened, partially closed or rotated independent of one another and are controlled electronically. By opening up a filtered aperture and closing off a non-filtered aperture SpO2 level can be reduced and vice versa.

In one embodiment both the first and second inlet filters 27, 37 partially restrict oxygen and allows other gases in air to pass freely from the entrances 28, 31 through the hollow mounting tube into breathing space in the airway mask 1. As the oxygen level in the user's blood slowly decreases, the user becomes drowsy and falls asleep. Since the inlet filters 27, 37 capacity to restrict oxygen inflow is partial, the inlet filters 27, 37 always permit enough oxygen to be admitted into the breathing space for safe sleeping. The inlet filters 27, 37 may be displaceable by removal of the valves or other parts, so as to allow cleaning or maintenance. The one-way inlet valves 22, 32 (valve only allows air in) which valves are in communication with the trigger event identification apparatus to be controlled according to identification of trigger events. The one-way inlet valves 22, 32 may be partially closed to hasten reduction of blood oxygen level. The inlet valves cannot be closed farther than is safe for sleeping.

The inlet valves 22, 32 are controlled by servomotors 24, 48. These are controlled in turn by the trigger event identification apparatus 26 with a transceiver, and a rechargeable battery 25. The trigger event identification apparatus 26 comprises an electronic circuit. Each inlet 22, 32, is operated individually be one of the servomotors.

In one embodiment both the first and second inlet filters 27, 37 favor passage of oxygen and restrict other gases in air from passing freely from the entrances 28, 31 through the hollow mounting tube into breathing space in the airway mask 1. As the oxygen level in the user's blood slowly increases, the user less drowsy and more alert. Since the inlet filters 27, 37 capacity to favor oxygen inflow is partial, the inlet filters 27, 37 always allow safe amount of oxygen to be admitted into the breathing space for living.

At least one of the one-way inlet valves 22, 32 may be partially closed to a first position to restrict the amount of oxygen allowed into the breathing space to that of the ambient air around user. At least one of the one-way inlet valves 22, 32 may be partially closed even further than the first position to a second position to restrict the amount of oxygen in the breathing level to make user's blood oxygen level to where they are drowsy, restful, and easily fall asleep. The inlet valves 22, 32 cannot be closed farther than is safe for sleeping. The trigger event identification apparatus operates the inlet valves according to identified trigger events to help the person sleep or wake up. The trigger event identification apparatus comprises a timer to schedule the person's sleep and wake cycle. The trigger event identification apparatus also uses the timer to identify trigger events that depend on timing and/or patterns of change in blood oxygen level and head orientation and movement.

The inlet filters 27, 37 comprise a semipermeable membrane which is selective for oxygen, and/or the inlet filters comprise a zeolite which selective absorbs oxygen or another gas in air such as carbon dioxide and/or nitrogen. In some embodiments the outlet valve 3 is coupled with an outlet filter 39 of membrane or zeolite type.

The airway mask 1 comprises the one-way outlet valve 3 in the nose and face cover. The one-way outlet valve 3 is disposed in an orifice 44 in the outer 12 and allows air to pass out of the breathing space within the airway mask 1. The outlet filter 39 is also disposed in the orifice 44. When the user breathes out, air leaves via the one-way valve 3, located centrally on the mask. This valve 3 may also be controlled digitally and adjust the oxygen proportion in the mask that has oxygen, carbon dioxide and nitrogen in according to identified trigger events.

The one-way outlet valve 3 can only be partially closed to safe limit so that person wearing the airway mask can exhaust enough breathed air out the mask to maintain a safe level of breathed air and oxygen within the breathing space.

The trigger event identification apparatus 26 is arranged to use feedback from the nasal oximeter 7 restrict or reduce or increase oxygen supply and to constantly or sufficiently frequently monitoring blood oxygen level with the nasal pulse oximeter 7 which is located in use at the bridge of the user's nose.

In this way the device adapted to be worn on a user's head to aid sleeping 99 is arranged to reduce the user's blood oxygen level to 90% to induce drowsiness and increase blood oxygen level to 95% when sleeping is detected. The trigger event identification apparatus 26 safely reduces the user's blood oxygen level in order to prevent insomnia and provide restful sleep. The orientation sensor 26 is monitored by the trigger event identification apparatus 26 so that various sleep states are identified as trigger events and the inlet valves 22, 32 and outlet valve 3 amount of opening is adjusted accordingly.

The pictured embodiment comprises a control PCB 26 with integrated 3 axial gyroscope accelerometer and gas probe and Li-po battery 25.

SpO2 level is monitored via the paranasal pulse oximeter wherein the gas probe within the mask also monitors the oxygen, carbon dioxide and nitrogen levels.

The device adapted to be worn on a user's head to aid sleeping 99 is operated using the user's SpO2 level throughout a wake, sleep, wake cycle. A single push button 36 is on the exterior of the cartridge 2 mounted to the front of the airway mask 1. Holding the button 36 urges the trigger event identification apparatus 26 to connect via Bluetooth® to an accompanying computer or smart phone.

An associated computer or smartphone application allows the user to set activation times for sleep and also tracks movement and thereby their sleep quality is also monitored. The app also includes advanced settings where the user can further calibrate the product performance within established safety parameters i.e. maximum and minimum SpO2 level, movement sensitivity etc.

The invention has been described by way of examples only and it will be appreciated that variation may be made to the above-mentioned embodiments without departing from the scope of invention as defined by the claims, in particular but not solely combination of features of described embodiments.

1. A device is adapted to be worn on a user's head to aid sleeping, the device comprises: a mask that defines part of an airway, the mask encloses a breathing space and includes at least one sensor which senses at least one trigger event and an oxygen regulator which is operative to vary an oxygen flow from an oxygen supply in response to a sensed at least one trigger event, wherein the sensed at least one trigger event causes the oxygen regulator to adjust an amount of oxygen in the breathing space.

2. The device according to claim 1 wherein the at least one sensor is an oximeter and the at least one trigger event is dependent upon a blood oxygen concentration level of the user.

3. The device according to claim 2 wherein the mask comprises a resiliently deformable portion and means to retain the mask on the user's head, the mask includes a bridge portion which bridges the user's nose, and the oximeter is retained in the bridge portion and is adapted to be pressed against a nose of user.

4. The device according to claim 1 wherein the at least one sensor includes a gas probe to measure levels of a gas within the breathing space, the gas being from the group comprising: argon, carbon dioxide, nitrogen and oxygen, and the at least one trigger event is dependent upon a concentration level of the measured level of gas.

5. The device according to claim 1 wherein at the least one sensor is an orientation sensor to detect an orientation of the user's head and the at least one trigger event is an orientation of the user's head which is associated with a risk factor.

6. The device according to claim 1 wherein the oxygen regulator comprises at least one filter is provided which has a semi-permeable membrane that is permeable to oxygen and non-permeable to at least one other gas which constitutes air.

7. The device according to claim 6 wherein the at least one filter comprises a zeolite material that is non-absorbent to oxygen and absorbs at least one other gas which constitutes air.

8. The device according to claim 6 wherein at least one filter is provided which has a semi-permeable membrane that is non-permeable to oxygen and permeable to at least one other gas which constitutes air.

9. The device according to claim 8 wherein the at least one filter comprises a zeolite material that absorbs oxygen and is does non absorb at least one other gas which constitutes air.

10. The device according to claim 6 wherein the at least one filter comprises a first filter in a first passage to filter ambient air into the breathing space in response to a trigger event.

11. The device according to claim 1 wherein the oxygen regulator comprises a first valve which is operative in response to a trigger event to regulate intake of filtered ambient air into the breathing space.

12. The device according to claim 11 wherein the first valve is a one-way valve that prevents flow of air out of the breathing space.

13. The device according to claim 12 wherein the first valve and a first filter are disposed in a first passage to filter ambient air to regulate flow of oxygen into the breathing space in response to a trigger event.

14. The device according to claim 6 wherein the at least one filter includes a second filter in a second passage to divert exhaled air from the breathing space in response to a trigger event.

15. The device according to claim 1 comprising a detachable cartridge which is affixed to a nose and a mouth cover of the airway mask, the cartridge comprising at least one filter and at least one sensor.

16. The device according to claim 15, wherein the cartridge further comprises a first valve to regulate intake of filtered ambient air into the breathing space in response to a trigger event, and/or comprising a second valve to regulate outflow of breathed air from the breathing space according to the trigger events.

17. The device according to claim 15 wherein the cartridge connects to a nose and mouth cover of the airway mask on a mounting which opens to allow the cartridge to receive ambient air external of the device.

18. The device according to claim 1 wherein the airway mask has a nose and mouth cover secured to an adjustable head strapping.
